(19) United States
(12) Patent Application Publication
(10) Pub. No.: US 2017/0165107 A1
(43) Pub. Date: Jun. 15, 2017

(54) HYPOTHERMIC PAIN-PREVENTING OR PAIN ALLEVIATING DEVICE

(71) Applicant: Cryo Devices, LLC, Boston, MA (US)

(72) Inventors: Samuel David Goos, Concord, MA (US); Joseph P. Lillis, Boston, MA (US)

(21) Appl. No.: 15/295,386

(22) Filed: Oct. 17, 2016

Related U.S. Application Data
(63) Continuation of application No. PCT/US2015/026125, filed on Apr. 16, 2015.
(60) Provisional application No. 61/981,179, filed on Apr. 17, 2014.

(51) Int. Cl. A61F 7/10; A61M 5/42

(52) U.S. Cl. CPC A61F 7/10; A61M 5/422; A61F 2007/108

(57) ABSTRACT

A hypothermic pain-preventing device and methods for preparation of human body surface before treatment are provided. The device includes a container with a liquid, gel or solid material and a plunger to displace the material from the container to the surface of body. The device can also be used to alleviate existing pain by applying the material of the container to the surface of body.

### RELATED APPLICATION

This application claims the benefit of priority of U.S. Provisional Application No. 61/981,179, entitled “Hypothermic Pain-Preventing or Pain Alleviating Device,” filed Apr. 17, 2014, the content of which is hereby incorporated by reference in its entirety.

### BACKGROUND OF DISCLOSURE

The present disclosure relates to tissue or body surface preparation, and more particularly, to devices and methods to prepare body surface or tissue surface before treatment.

Gating theory has proposed that perception of pain may be greatly alleviated by providing alternative sensation such as pressure, vibration, or temperature that can make the brain unable to fully process the pain signals.

The concept of preventing pain by reducing temperature of skin/tissue using cold materials is well known. Chilling reduces pain, bleeding and swelling of treatment or procedure. Rubbing ice provides fast reduction of skin temperature, and sterilization provides the safest way to use on skin and mucous membranes. In addition, chilling the skin or tissue after treatment reduces swelling and bruising. Applying this device and method can greatly alleviate the perception of pain before treatment and reduces the swelling and bruising after treatment. Therefore, providing a more comfortable procedure for treatment or introduction of anesthetic to tissue or body surface.

Accordingly, the present disclosure provides method and device for preparing body surface or tissue surface to prevent pain before treatment or as a treatment for exiting pain.

### SUMMARY

According to certain embodiments, a hypothermic pain-preventing device for preparing body or tissue surface before treatment is provided. The device can comprise a container having a bottom base and an open top, configured to contain a liquid, gel or solid material, and a removable cap which is positioned on the open top of the container. The device can further comprise at least one material inside the container, wherein the material is liquid, gel or solid and a plunger positioned on the bottom base of the container, configured to displace the at least one material inside the container.

Preparing body or tissue surface before treatment comprises preparing the skin or mucous membrane before treatment. In one embodiment, preparing skin before treatment entails numbing the skin before treatment. The treatment can be selected from the group consisting of injection, aspiration and stereotactic biopsy.

The container may have a cylindrical shape and it may be flexible. In one embodiment, the container is made of polypropylene.

The material in the container can comprise purified, sterilized or distilled water in liquid or solid form. The material in the container can further comprise an anesthetic. Also, the material in the container can comprise a sterilization agent, for example, Povidone-iodine, Chlorhexidine, Chlorhexidine Gluconate, Sodium Chloride Iodine, Providone-iodine, Triclosan, Hydrogen peroxide or Isopropyl alcohol.

In one embodiment, the material in the container can be frozen.

The area of the bottom base of the container is larger than the area of the open top of the container.

The device can further comprise a gasket on the bottom base to prevent leakage of material from the container.

According to other embodiments, the invention provides a hypothermic pain-preventing method for preparing a body or tissue surface before treatment is provided. The method can comprise selecting part of a tissue to be treated and placing a container having a bottom base and an open top that contain a liquid, gel or solid material on the tissue. The method can further comprise removing a cap positioned on the open top of the container and operating a plunger positioned on the bottom base of the container to displace the material inside the container. In one embodiment of the method, the container comprises a frozen aqueous material that numbs the body or skin surface when applied.

### BRIEF DESCRIPTION OF THE DRAWINGS

FIG. 1 illustrates a device for preparing a body surface before treatment, according to certain embodiments.

FIG. 2 illustrates a device for preparing a body surface before treatment, according to certain embodiments.

### DESCRIPTION OF EXEMPLARY EMBODIMENTS

Reference will now be made in detail to certain exemplary embodiments according to the present disclosure, certain examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless specifically stated otherwise. Furthermore, the use of the term “including”, as well as other forms, such as “includes” and “included”, is not limiting. Any range described herein will be understood to include the endpoints and all values between the endpoints.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirely for any purpose.

A “hypothermic pain-preventing device” means a device for preventing pain before treatment to body or tissue surface.

A material inside the container can be in three phases. The material can be in liquid phase or it can also be in a solid phase. In addition, the material can be in transition from liquid phase to solid phase. Therefore, this phase of the material will be referred to as a gel.

The device includes a plunger, also referred to herein as a slide mechanism. A plunger is configured to displace a material which is inside the container. The “plunger” refers to a part that moves up and down inside a container to push material out of the container. The plunger can also retract materials inside the container.

The material in the container may include an anesthetic. “Anesthetic” refers to a substance that induces insensitivity of surface of body or tissue to pain.

The material in the container can further include sterilizer. “Sterilizer” refers to a material that has the ability to destroy bacteria or germs.

Numb refers to the situation that the skin is deprived from physical sensation.

The present disclosure relates to tissue or body surface preparation, and more particularly, to devices and methods for preparing body surface or tissue surface before treatment. Before performing any treatment to body surface, it is preferred to alleviate the perception of pain or prevent pain before treatment. Also, it is more desirable to reduce the existing pain which is resulted from skin burns or shin splint condition or even to reduce the swelling and bruising of the body surface or tissue after the treatment. In addition, sterilizing the body surface or tissue while preventing pain before treatment is more desirable. This can be done by reducing temperature of body surface or tissue using cold materials. These treatments can be, but not limited to, injection, aspiration, transcutaneous, percutaneous and stereotactic biopsies. For example, injection of anesthetics, injection of medications, needle thyroid aspiration, lung aspiration, liver aspiration, pericardium and kidney aspiration, spinal tap aspiration, stereotactic needle biopsy in radiology for breast procedures, injection of marking dyes prior to sentinel node biopsy, injecting cosmetic fillers or Botox, placement of J tube, bladder taps, placement of intravenous and intrarterial catheters, spinal catheterization, preparation for joint injection, preparation for arthrocentesis, preparation for transrectal and perineal prostate biopsy.

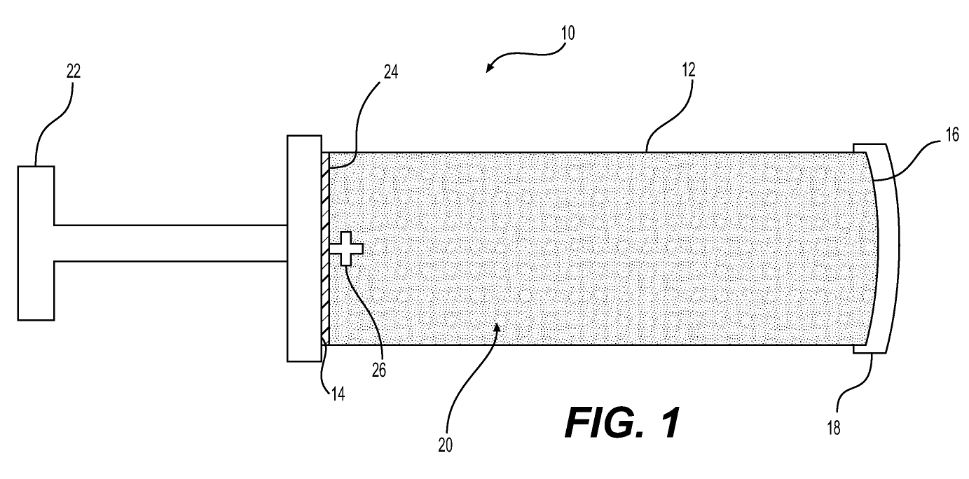

FIG. 1 illustrates a device 10 for preparing body surface or tissue surface before treatment according to certain embodiments. As shown, the device 10, a hypothermic pain-preventing device, can comprise a container 12 having a bottom base 14 and an open top 16, configured to contain a liquid, gel or solid material, and a removable cap 18 which is positioned on the open top 16 of the container 12.

The device 10 can further comprise at least one material 20 inside the container 12, wherein the material 20 is liquid, gel or solid. Also, the device 10 can comprise a plunger 22 positioned on the bottom base 14 of the container 12, configured to displace the at least one material 20 inside the container 12.

The container 12 can be constructed from a variety of different materials. The container 12 can be made of a rigid material such as rigid plastic, or a flexible material. The flexible material can be any kind of polymers such as polypropylene. The container 12 should withstand autoclave. The container 12 can be made in any shape such as cylindrical, cubical or any other geometrical shape. In general, the container 12 is configured to contain material 20 in any form of liquid, gel or solid. The container 12 can contain liquid and then be placed in a freezer, refrigerator or any equipment that can reduce the temperature of the material 20, liquid in this case, below freezing point. Therefore, the container 20 should withstand low temperature and preferably not change shape at macro level when its surrounding environment's temperature changes from room temperature to its contained material freezing point. For example, if the container 12 contains water at room temperature, it shouldn't change shape at macro level when it is placed in an environment that the temperature changes from −50° C. to 0° C.

As noted above, the device 10 can further include at least one material 20 inside the container 12, wherein the material 20 can be liquid, gel or solid. The material 20 can be made of only a single substance or a combination of different substances. The material 20 in the container can include purified or distilled water in liquid or solid form. The material in the container can further contain an anesthetic. The advantage of sterile water is that it is not toxic to human mucous. For example, sterile water is not toxic to membranes or wounds.

The container 12 will be placed on surface of body or tissue before treatment. Therefore, the material 20 will be in contact with surface of body or tissue to prepare the surface of body or tissue for treatment. In some embodiments, preparing surface of body or tissue before treatment comprises preparing the skin before treatment. In some embodiments, preparing skin before treatment is configured to numb skin before treatment.

In some embodiments, the material 20 inside container 12 can include anesthetic such as betadine to prepare a sterile body surface or tissue prior to treatment. The betadine freezes at a lower temperature than water. Therefore, other materials can be used to control the temperature of the material 20 inside the container 12 for temperature sensitive treatment.

In some embodiments, the material 20 can include, but not limited to, Povidone-Iodine (PVP-I), Chlorhexidine-topical, Chlorhexidine, Chlorhexidine Gluconate, Sodium Chloride, Iodine, Providone-Iodine, Triclosan, Hydrogen peroxide, Isopropyl alcohol.

As noted, the container 12 has a bottom base 14 and an open top 16. In addition, the device 10 can include a removable cap 18 which is placed on the open top 16. The removable cap 18 can be made of any material and it can be in any shape. The removable cap 18 should withstand autoclave as well. The removable cap 18 can further allow for any expansion of material 20 inside the container 12 as well as for the displacement of air or space within the container 12. This can be lead to more consistent freezing and/or solidification of the material 20 inside the container 12 at either a reduced or increased temperature or pressure.

As noted above, the device 10 can comprise a plunger 22 positioned on the bottom base 14 of the container 12, configured to displace the at least one material 20 inside the container 12. The plunger 22 can be made of any material. The plunger 22 can be made of polymers; for example polypropylene. Further, the plunger 22 should be made of materials that can withstand autoclave. Depending on the shape of the container 12, the plunger 22 should be made such that it will fit into the container 12 when displacing material 20 inside the container 12.

In some embodiments, the bottom base 14 can include a fastening/gripping barb 26 which will allow for adherence of the material 20 to the bottom base 14 during physical state change, changing from liquid to gel or solid, and also allow for the bottom base 14 to retract the material 20 back inside to the container 12 when needed.

In some embodiments, the bottom base 14 of the container 12 will be outfitted with a slide mechanism which allows for the extension and retraction of the material 20 to be applied to body surface or tissue when the cap 18 is removed to allow for device 10 to be set down during treatment without compromising solid or gel material 20 for continued use of body surface or tissue of the same patient.

The bottom base 14, can further include a gasket 24 to prevent leakage of liquid or gel phase of material 20 inside the container 12. The gasket 24 is capable of withstanding low temperatures and expansion or contraction of container 12 and/or material 20.

In some embodiments, the bottom base 14 of the container 12 can have a greater diameter than the open top 16, i.e. capped top. This will allow the material 20 of the device 10, when laid down horizontally, to pitch toward the open top 16 of the container 12. Therefore, forces the material 20, in liquid or gel form, to uniformly fill the cap 18 and force out any small pockets of air or other gas to the bottom base 14 of the container 12.

As noted above, the whole device 10, including container 12, removable cap 18, plunger 22, gasket 24, fastening/gripping barb 26 and any additional parts that define the device 10 should withstand autoclave.

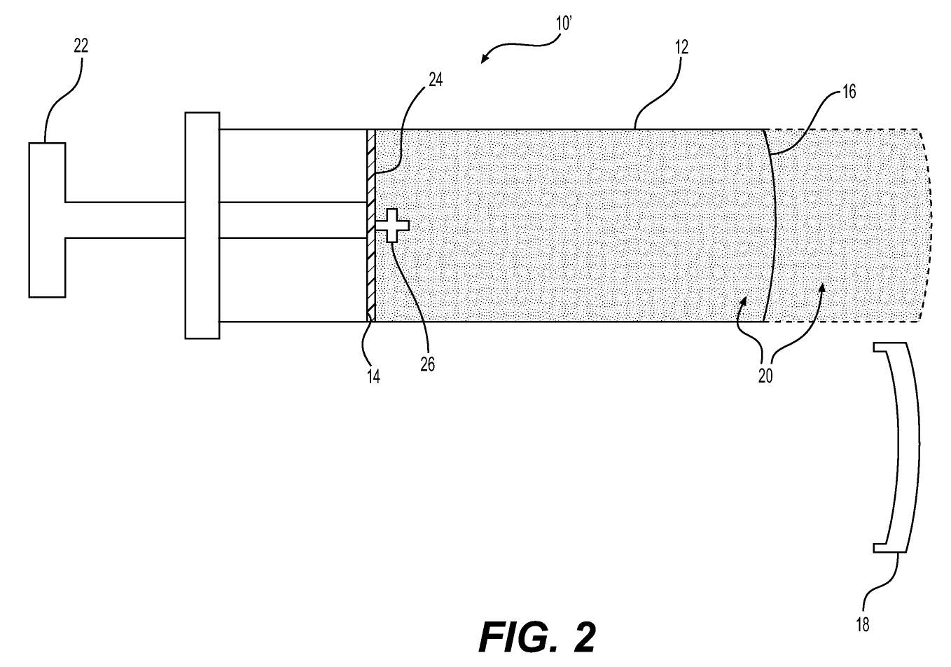

FIG. 2 illustrates a device 10 for preparing body surface or tissue surface before treatment according to certain embodiments wherein cap 18 is removed to allow exposure of material 20 to body or tissue surface.

1. A hypothermic pain-preventing device for preparing body or tissue surface before treatment comprising:
a container having a bottom base and an open top, configured to contain a liquid, gel or solid material;
a removable cap positioned on the open top of the container;
at least one material inside the container, wherein the material is liquid, gel or solid; and
a plunger positioned on the bottom base of the container, configured to displace the at least one material inside the container.

2. The device of claim 1, wherein preparing body or tissue surface before treatment comprises preparing the skin before treatment.

3. The device of claim 2, wherein preparing skin before treatment is configured to numb the skin before treatment.

4. The device of claim 2, wherein treatment is selected from the group consisting of injection, aspiration and stereotactic biopsy.

5. (canceled)

6. The device of claim 2, wherein the container is flexible.

7. (canceled)

8. The device of claim 2, wherein the material in the container comprises purified or distilled water in liquid or solid form, an anesthetic, or a sterilizer.

9. (canceled)

10. (canceled)

11. The device of claim 2, wherein the material in the container comprises Povidone-iodine, Chlorhexidine, Chlorhexidine Gluconate, Sodium Chloride Iodine, Providone-iodine, Triclosan, Hydrogen peroxide or Isopropyl alcohol.

12. The device of claim 2, wherein the material in the container is frozen.

13. The device of claim 2, wherein the area of the bottom base of the container is larger than the area of the open top of the container.

14. The device of claim 2, further comprises a gasket on the bottom base to prevent leakage of material from the container.

15. A hypothermic pain-preventing method for preparing body or tissue surface before treatment comprising:
selecting part of a tissue to be treated;
placing a container having a bottom base and an open top that contain a liquid, gel or solid material on the tissue;
removing a cap positioned on the open top of the container; and
operating a plunger positioned on the bottom base of the container to displace the material inside the container.

16. The method of claim 15, wherein preparing body or tissue surface before treatment comprises preparing skin before treatment.

17. The method of claim 16, wherein preparing skin before treatment is configured to numb the skin before treatment.

18. The method of claim 16, wherein treatment is selected from the group consisting of injection, aspiration and stereotactic biopsy.

19. (canceled)

20. The method of claim 16, wherein the container is flexible.

21. (canceled)

22. The method of claim 16, wherein the material in the container comprises purified or distilled water in liquid or solid form, an anesthetic, or a sterilizer.

23. (canceled)

24. (canceled)

25. The method of claim 16, wherein the material in the container comprises Povidone-iodine, Chlorhexidine, Chlorhexidine Gluconate, Sodium Chloride Iodine, Providone-iodine, Triclosan, Hydrogen peroxide or Isopropyl alcohol.

26. The method of claim 16, wherein the material in the container is frozen.

27. The method of claim 16, wherein the area of the bottom base of the container is larger than the area of the open top of the container.

28. The method of claim 16, further comprises a gasket on the bottom base to prevent leakage of material from the container.
